(19) United States
(12) Patent Application Publication
(10) Pub. No.: US 2005/0049465 A1
(43) Pub. Date: Mar. 3, 2005

(54) Apparatus monitoring signal in situ

(76) Inventor: Wei-Kung Wang, Taipei (TW)

Correspondence Address: Ladas & Parry, 26 West 61th Street, New York, NY, 10023, US

(21) Appl. No.: 10/924,021

(22) Filed: Aug. 23, 2004

(30) Foreign Application Priority Data
Aug. 27, 2003 (TW) 092123724

(51) Int. Cl. A61B 5/00

(52) U.S. Cl. 600/309; 600/310; 600/322

(57) ABSTRACT

For repeatedly measuring signals from the same part of the tissue to monitor the blood composition, we use elastic membranes at upper and lower parts of the intruded tissue together with a cone-shaped guide. This will constrain the tissue in the same place when the signal guide is used for measuring the signals from the same part of tissue repeatedly. The signals can be from the aggregate of the designated composition with the other ingredients of the blood. For example, we may use the signals from the precursor of Glycated hemoglobin (HbAlc) to measure the concentration of blood glucose. For bio-secure purpose, the cone-shaped guide will be extended to cover tightly most part of the finger.

### BACKGROUND OF THE INVENTION

(A) Field of the Invention

This invention relates to medical devices, blood composition sampling and a bio-secure system.

(B) Description of Related Art

U.S. application Ser. No. 10/123,124 and U.S. application Ser. No. 10/207,610

### SUMMARY OF THE INVENTION

The most important physiological parameters, such as blood sugar, blood oxygen and cholesterol, need to be repeatedly measured in order to monitor the variations thereof. This invention is a CIP application of U.S. patent application Ser. No. 10/123,124, entitled “Method and Apparatus for Measuring Concentration of a Specific Ingredient in Situ”, filed by the same applicant. For such a purpose, it is provided with a signal source in the tissue, for example, radio-isotope in blood will emit signals such as α, β, γ particles. Also, the signals can be electromagnetic wave (visible light, UV, IR, X-ray, microwave) from outside the body. After the tissue is irradiated, absorption, scattering, fluorescence, etc., are induced in the tissue. A signal analyzer or a spectrum analyzer may be used to monitor the concentration of ingredients in the tissue. This signal may not be from the ingredient itself and can be an aggregate of the ingredient with some other specific component, such as
Aggregate⇄ingredient (to be measured)+specific component.

In the invention, glucose and hemoglobin are used as an example.
Hemoglobin+glucose⇄HbAlc (precursor)⇄HbAlc

Before hemoglobin and glucose become a compound HbAlc, there is an intermediate stage of aggregate HbAlc (precursor). As the concentration of Hb is somewhat stable, we may figure out the concentration of glucose in the blood from the signal of HbAlc (precursor).

To fix the tissue at the same position for sequential measurements, a tissue adaptor is invented. This adaptor will work better with an extrude tissue, such as a finger or toe. If there is a cone shape guide inside the adaptor, it will guide the fingers to anchor at the top of the cone in operation. To secure the fingers in a stretched position and at the right angle, soft pads both above and below the fingers can be used. These soft pads could be replaced by elastic membranes which are stretched to wrap around the fingers and hold the fingers smoothly and softly so that no blood circulation is interfered. These pads or membranes can be in a concaved structure. This entire structure is called the tissue adaptor. Springs are used to hold the tissue adaptor to improve its adaptability.

To improve the precision of positioning the fingers, a patient's palm may be placed on a flat pad, so that the fingers will not rotate due to incorrect posture. For security purposes, such as an application with U.S. patent application Ser. No. 10/207,610, entitled “Biosecure Method and Device CIP,” the cone of the tissue adaptor extends to cover some or even most part of a finger and to fit tightly with the finger, so that fingers with different shapes cannot fit into the shape. If a finger is too large, it cannot enter the designated slot, and if too small, it cannot be stable.

### BRIEF DESCRIPTION OF THE DRAWINGS

FIG. 1 is a cross-sectional view of an embodiment of the invention.

### DETAILED DESCRIPTION OF THE INVENTION

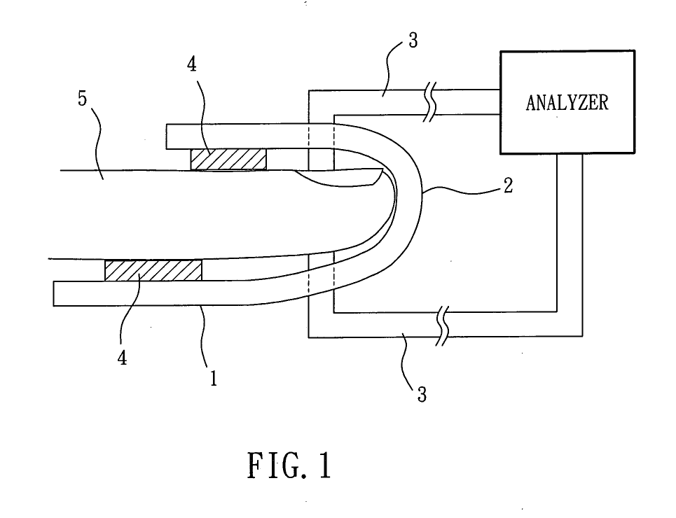

Referring to FIG. 1, a concaved slot 1 is made by a mold in the shape of an extrude tissue, here a finger 5. The slot 1 and the finger 5 are complementary in shape. Soft pads 4 are located inside the slot and both above and below the finger 5 so as not to interfere signals. A cone-shaped guide 2 is located at the distal end inside the slot to insure the correct position of the finger 5. The guide 2 has an additional signal guide device 3 which transmits signals to the tissue, here a finger 5, and transmits modulated signals back to the device 3 so as to acquire signals in situ repeatedly. It is further pointed out by the invention that the signals used for composition analysis of a special ingredient are generated by said ingredient-making chemical actions with other ingredients in blood. If used in bio-secure purposes, the concaved slot 1 and the extruded tissue contact each other tightly to achieve the purpose of lock and key. The said bio-secure function could be an additional security function other than spectrum identification.

1. An apparatus for monitoring a signal from a tissue in situ comprising:
a tissue adaptor to fix the tissue at the same place during repeated measurements;
a signal producer; and
a signal analyzer;
wherein the signal produced by the signal producer is interacted with the tissue and then be measured by the signal analyzer.

2. An apparatus as claimed in claim 1, wherein the said signal producer comprises a signal generator outside the tissue and interacts with the tissue to create the signal producer.

3. An apparatus as claimed in claim 1, wherein the said signal producer is in the tissue.

4. An apparatus as claimed in claim 1, wherein the tissue comprises an extrude shape.

5. An apparatus as claimed in claim 4, wherein the said extrude shape comprises a finger.

6. An apparatus as claimed in claim 1, wherein the said tissue adaptor comprises a soft pad.

7. An apparatus as claimed in claim 6, wherein the said soft pad is above the tissue.

8. An apparatus as claimed in claim 6, wherein the said soft pad is below the tissue.

9. An apparatus as claimed in claim 4, further comprising a cone shaped guide to confine the tissue.

10. An apparatus as claimed in claim 4, further comprising a concaved structure fit closely to the extruded tissue.

11. An apparatus as claimed in claim 2, wherein the said signal outside the tissue comprises electromagnetic wave.

12. An apparatus as claimed in claim 1, wherein the said signal producer comprises an ingredient in the tissue.

13. An apparatus as claimed in claim 12, wherein the said ingredient comprises an aggregate.

14. An apparatus as claimed in claim 13, wherein the said aggregate comprises glucose.

15. An apparatus as claimed in claim 13, wherein the said aggregate comprises hemoglobin.

16. An apparatus as claimed in claim 13, wherein the said aggregate comprises glucose and hemoglobin.

17. An apparatus as claimed in claim 1, wherein the said tissue adaptor comprises a flat area.

18. An apparatus as claimed in claim 1, wherein the said tissue adaptor comprises a spring.

19. An apparatus as claimed in claim 1, wherein the said tissue adaptor comprises an elastic membrane.

20. An apparatus as claimed in claim 1, wherein the said elastic membrane is above the tissue.

21. An apparatus as claimed in claim 1, wherein the said elastic membrane is below the tissue.

22. An apparatus for measuring a specific ingredient in blood comprises:
a signal generator; and
an analyzer to analyze the signal from the aggregate of said ingredient to determine the concentration of said ingredient.

23. An apparatus as claimed in claim 22, wherein the said blood comprises the signal generator.

24. An apparatus as claimed in claim 23, wherein said signal generator is generated from a signal from outside the blood and interactes with the blood to generate the signal.

25. An apparatus as claimed in claim 22, wherein the said specific ingredient comprises glucose.

26. An apparatus as claimed in claim 25, wherein the said glucose aggregate comprises hemoglobin.

27. An apparatus as claimed in claim 22, wherein the said signal from said aggregate comprises electromagnetic wave.

28. An apparatus as claimed in claim 22, wherein the said signal from the said aggregate comprises radiations.

29. An apparatus as claimed in claim 9, wherein the cone shaped guide is extended to cover tightly some part of the finger.
